(19) United States
(12) Patent Application Publication
(10) Pub. No.: US 2014/0276045 A1
(43) Pub. Date: Sep. 18, 2014

(54) METHOD AND APPARATUS FOR PROCESSING ULTRASOUND DATA USING SCAN LINE INFORMATION

(71) Applicant: SAMSUNG ELECTRONICS CO., LTD., Suwon-si (KR)

(72) Inventors: Young-yoon LEE, Yongin-si (KR); Hyun-taek LEE, Sejong (KR); Hee-chul YOON, Seoul (KR); Jin-woo YIM, Seongnam-si (KR); Young-ho MOON, Suwon-si (KR); Kiwon SOHN, Seoul (KR); Hae-kyung JUNG, Seoul (KR)

(73) Assignee: SAMSUNG ELECTRONICS CO., LTD., Suwon-si (KR)

(21) Appl. No.: 14/102,669

(22) Filed: Dec. 11, 2013

(30) Foreign Application Priority Data
Mar. 15, 2013 (KR) 10-2013-0028246

(51) Int. Cl. A61B 8/08; A61B 8/00

(52) U.S. Cl. CPC A61B 8/5269; A61B 8/461
USPC 600/437

(57) ABSTRACT

A method of and apparatus for processing ultrasound data acquired from an object is provided. With the methods and apparatuses of the exemplary embodiments, speckles in an ultrasound image are reduced by using scan line information of an ultrasound signal applied to the object prior to scan conversion of the ultrasound image.

### CROSS-REFERENCE TO RELATED PATENT APPLICATION

This application claims the benefit of Korean Patent Application No. 10-2013-0028246, filed on Mar. 15, 2013, in the Korean Intellectual Property Office, the disclosure of which is incorporated herein by reference, in its entirety.

### BACKGROUND

1. Field

The exemplary embodiments relate to a method and apparatus for processing ultrasound data to improve the quality of an image generated by using the scan line information related to a transmitted ultrasound signal.

2. Description of the Related Art

Ultrasound diagnosis devices irradiate an ultrasound signal (generally having a frequency of 20 KHz or above) to a predetermined part inside an object by using a probe and may acquire an image of the predetermined part by using a reflected echo signal. In particular, the ultrasound diagnosis devices are used for medical purposes, such as detection of foreign substances, injury measurement, medical observation, and so forth, inside an object. The ultrasound diagnosis devices are widely used together with other image diagnosis devices because of advantages such as high stability, real-time display capability and safety from radiation exposure.

An ultrasound image acquired by an ultrasound diagnosis device may be displayed on the ultrasound diagnosis device or may be stored in a storage medium and displayed on another image display device. For example, the ultrasound image may be reduced and displayed on a screen of a portable phone, a portable electronic device, a personal digital assistant (PDA), a tablet PC, or the like.

Noise due to interference between ultrasound signals may appear in an ultrasound image, for example, as scattered dots called speckles. Accordingly, a strong need arises for efficiently removing noise from an ultrasound image.

### SUMMARY

The exemplary embodiments provide a method and apparatus for efficiently improving the quality of an image which corresponds to ultrasound data by removing noise when the image is generated by processing the ultrasound data.

The exemplary embodiments also provide a non-transitory computer-readable storage medium having stored therein program instructions, which when executed by a processor of a computer, causes the computer to perform the method.

According to an aspect of the exemplary embodiments, there is provided a method of processing ultrasound data, the method including: acquiring an ultrasound image related to an object by applying an ultrasound signal to the object; acquiring scan line information related to the ultrasound signal applied to the object; and reducing speckles of the ultrasound image by using the scan line information before scan conversion of the ultrasound image.

The reducing speckles of the ultra sound image may include detecting an edge in the ultrasound image by using the scan line information.

The acquiring scan line information may include acquiring at least one of a strength and a direction of the detected edge.

The reducing may include processing ultrasound images in regions divided by the detected edge, based on different references.

The processing may include processing the ultrasound image in the divided regions based on the direction of the detected edge.

The detecting may include determining an asymmetrical window of a predetermined size to be applied to the ultrasound image based on the scan line information; and detecting the edge by using the asymmetrical window.

The determining of the asymmetrical window may include determining a horizontal length and a vertical length of the asymmetrical window.

The detecting may include down-sampling the ultrasound image based on the scan line information; and detecting an edge of the down-sampled ultrasound image.

The method may further include: scan-converting the ultrasound image from which the speckles have been reduced; and displaying a scan-converted ultrasound image.

According to another aspect of the exemplary embodiments, an apparatus is provided for processing ultrasound data, the apparatus including: a data acquisition device configured to acquire an ultrasound image related to an object by applying an ultrasound signal to the object; a scan line information acquisition device configured to acquire scan line information related to the ultrasound signal applied to the object; and a data processor configured to reduce speckles of the ultrasound image by using the scan line information prior to scan conversion of the ultrasound image.

An aspect of an exemplary embodiment may provide an apparatus for processing ultrasound data, the apparatus including: a scan line information acquisition device configured to acquire scan line information related to an ultrasound image; and a data processor configured to reduce speckles of the ultrasound image by using the scan line information prior to scan conversion of the ultrasound image.

The apparatus for processing ultrasound data may further include a data acquisition device configured to acquire an ultrasound image related to an object by applying an ultrasound signal to the object.

The apparatus for processing ultrasound data may further include a scan converter configured to scan-convert a ultrasound image from which the speckles have been reduced. In addition, the apparatus for processing ultrasound data of claim 22, further comprising a display configured to display a scan-converted ultrasound image.

The data processor may be configured to detect an edge included in the ultrasound image by using the scan line information. The data processor may be configured to acquire at least one of a strength and a direction of the detected edge.

Moreover, the data processor may be configured to process an ultrasound image in regions divided by the detected edge based on different references.

According to another aspect of the exemplary embodiments, there is provided a non-transitory computer-readable storage medium having stored therein program instructions, which when executed by a processor for computer, causes the computer to perform a method of processing ultrasound data.

### BRIEF DESCRIPTION OF THE DRAWINGS

The exemplary embodiments will be readily understood from a combination of the detailed description below and their accompanying drawings, wherein reference numerals denote structural elements.

The above and other features and advantages will become more apparent by describing in detail exemplary embodiments thereof with reference to the attached drawings in which:

FIG. 1 is a block diagram for describing a method of processing ultrasound data, according to the related art;

FIG. 2 is a block diagram of an apparatus for processing ultrasound data, according to an exemplary embodiment;

FIG. 3 is a flowchart which illustrates a method of processing ultrasound data, according to an exemplary;

FIG. 4 is a flowchart which illustrates a method of processing ultrasound data, according to another exemplary embodiment;

FIG. 5 illustrates a scan conversion process according to an exemplary embodiment;

FIG. 6 illustrates a process of processing regions divided by a detected edge, according to an exemplary embodiment;

FIG. 7 illustrates a process of setting a window to be applied to ultrasound data, according to an exemplary embodiment; and

FIG. 8 illustrates a process of down-sampling and processing ultrasound data, according to an exemplary embodiment.

### DETAILED DESCRIPTION OF THE EXEMPLARY EMBODIMENTS

Although general terms are used in the exemplary embodiments while taking the functions in the exemplary embodiments into account, the terms may vary according to the intention of one of ordinary skill in the art, case precedents, or the appearance of new technology. In addition, in specific cases, terms intentionally selected by the applicant may be used. In this case, the meaning of the terms will be disclosed in the corresponding description of the exemplary embodiments. Accordingly, the terms used in the exemplary embodiments should be defined not by their simple names but by their meanings and contents.

In the specification, when a certain part “includes” a certain component, this indicates that the part may further include another component instead of excluding another component unless there is different disclosure. In addition, the term, such as “ . . . device” or “ . . . module,” disclosed in the specification indicates a unit for processing at least one function or operation, and this may be implemented by hardware, software, or a combination thereof.

As used herein, expressions such as “at least one of,” when preceding a list of elements, modify the entire list of elements and do not modify the individual elements of the list.

In the specification, the term “ultrasound image” indicates an image of an object which is acquired by using ultrasound waves. In addition, the term “object” may include a human being, an animal, or a part of the human being or the animal. For example, the object may be a blood vessel, a blood flow, a tissue, a bone, or an organ such as the liver, the heart, the womb, the brain, the breast, the abdomen, or the like. In addition, the object may be a phantom that has almost the same volume, density, and effective atomic number as an organ or a spherical water phantom having similar properties to the human body.

In addition, in the specification, the term “user” denotes a medical expert such as a medical practitioner, a nurse, a radiographer, a clinical pathologist, a medical image expert, or the like or a service engineer for repairing medical equipment, but is not limited thereto.

Hereinafter, exemplary embodiments will be described in detail with reference to the drawings.

FIG. 1 is a block diagram which describes a method of processing ultrasound data, according to the related art.

In operation 12, when an ultrasound signal transmitted to an object is reflected from the object, an ultrasound diagnosis device receives a reflected ultrasound signal that is referred to as an echo signal. The ultrasound diagnosis device may transmit an ultrasound signal through a plurality of transducers included in a probe and receive an echo signal from an object.

In operation 14, the ultrasound diagnosis device generates ultrasound data by digitizing the echo signal and focusing the digitized echo signal by using a digital beam former.

In operation 16, the ultrasound diagnosis device performs scan conversion of the generated ultrasound data. The scan conversion is a process of converting the ultrasound data to an ultrasound digital image, i.e., a process of converting the ultrasound data to an ultrasound digital image characterized by various coordinates.

In operation 18, the ultrasound diagnosis device improves the image quality of the scan-converted ultrasound digital image. For example, the ultrasound diagnosis device may improve the image quality of the ultrasound image by removing speckles from the ultrasound image.

In operation 20, the ultrasound diagnosis device displays the ultrasound image with improved image quality. As described above, the ultrasound diagnosis device according to the related art performs image quality improvement processing on the scan-converted ultrasound image. However, in response to noise of the scan-converted ultrasound image being removed, the noise, such as speckles, may not be efficiently removed due to wide spreading in a lateral direction.

FIG. 2 is a block diagram of an apparatus 100 for processing ultrasound data, according to an exemplary embodiment. The apparatus 100 may include a data acquisition device 110, a scan line information acquisition device 120, a data processor 130, a scan converter 140, and a display 150. However, the configuration of the apparatus 100 is not limited to the configuration shown in FIG. 2, and the apparatus 100 may further include other general-use components.

The apparatus 100 generates an ultrasound image by processing an ultrasound signal acquired by scanning an object 50 and provides the generated ultrasound image to a user. The ultrasound image may not only be a two-dimensional (2D) image showing a cross-sectional surface of the object 50 but may also be a 3D image. In addition, the ultrasound image may not only be a gray scale ultrasound image acquired by scanning the object 50 in an amplitude mode (A mode), a brightness mode (B mode), or a motion mode (M mode) but also a Doppler image in which a motion of the object 50 is represented by colors by using Doppler data.

The apparatus 100 may not only provide an ultrasound image generated by directly scanning the object 50 but may also provide ultrasound data received from an external device or a server connected over a network. In the former case, the apparatus 100 may be implemented as a fixed-type/movable-type ultrasound diagnosis device. In the latter case, the apparatus 100 may be implemented in various forms including a screen on which ultrasound information is to be provided.

For example, the apparatus 100 may be implemented in various forms including a screen capable of outputting an image thereon, such as a work station, a picture archiving and communication system (PACS) viewer, a portable phone, a smart phone, a smart TV, an Internet protocol TV (IPTV), a digital TV (DTV), a personal computer (PC), a laptop computer, a tablet PC, an e-book terminal, a personal digital assistant (PDA), a portable multimedia player (PMP), a navigation, a consumer electronics (CE) device, and the like.

The data acquisition device 110 acquires ultrasound data related to the object 50. The data acquisition device 110 may acquire ultrasound data by scanning the object 50 through a probe and processing an echo signal. In addition, the data acquisition device 110 may receive ultrasound data from an external device or a server connected over a network in a wired or wireless manner.

In response to the data acquisition device 110 acquiring ultrasound data by scanning the object 50, one or more probes may be used. That is, the data acquisition device 110 may acquire ultrasound data by using various types of probes, such as a linear array probe, a convex array probe, a phased array probe, a matrix probe, and so forth.

When the data acquisition device 110 scans the object 50, the data acquisition device 110 may acquire ultrasound data along a scan line. The scan line indicates a path along which an ultrasound signal transmitted from a transducer is focused. That is, a transmission ultrasound signal is focused on the object 50 while moving along a scan line and reflected from the focused point. When a reception ultrasound signal (echo signal) is acquired along one scan line, the transmission ultrasound signal is focused along a new scan line. The data acquisition device 110 may acquire ultrasound data of the object 50 by combining reception ultrasound signals acquired along a plurality of scan lines.

When the data acquisition device 110 acquires ultrasound data through a network, various types of communication means may be used. For example, the data acquisition device 110 may be connected to a network through a wired connection with a universal serial bus (USB), a data cable, an optical fiber cable, or the like or a wireless connection with Bluetooth®, Wi-Fi, near field communication (NFC), a 2G/3G/4G network, or the like. The data acquisition device 110 may acquire ultrasound data from an external device or a cloud server by being connected to a network. The data acquisition device 110 may acquire ultrasound data from another device in a hospital server or a server connected to a PACS.

The scan line information acquisition device 120 acquires scan line information that is information regarding a transmission ultrasound signal. The scan line information may include information related to the scan lines described above and related information between a scan line and the transmission ultrasound signal. For example, the scan line information may include information related to a depth for focusing an ultrasound signal, i.e., a depth of the transmission ultrasound signal on a scan line. In addition, the scan line information may include information regarding a gap between paths on which the ultrasound signal is focused, i.e., information regarding a gap between scan lines. As another example, the scan line information may include information regarding a shape of scan lines that are paths on which the ultrasound signal is transmitted.

According to an exemplary embodiment, the scan line information may not only include information regarding the scan lines but also information regarding a probe and a transducer. For example, the scan line information may include information regarding various types of probe, information regarding pulses of an ultrasound signal transmitted by a transducer, information regarding a transmission ultrasound signal delayed by a beam former, and so forth.

The scan line information acquisition device 120 may receive and acquire scan line information from the data acquisition device 110. In response to the data acquisition device 110 generating the ultrasound data by directly scanning the object 50, the scan line information acquisition device 120 may acquire information relating to scan lines used during a process of scanning the object 50. When the data acquisition device 110 receives the ultrasound data over a network, the scan line information acquisition device 120 may extract and acquire information regarding scan lines from the ultrasound data.

The data processor 130 processes the ultrasound data by using the scan line information. The data processor 130 may improve image quality of an ultrasound image by reducing speckles of the ultrasound image before scan conversion of the ultrasound image data by the scan converter 140.

In particular, the data processor 130 may detect an edge included in the ultrasound image before the scan conversion by using the scan line information. The data processor 130 may check a structure of the object 50 by detecting an edge of an organ, bone, blood vessel, tissue, or the like from the ultrasound image prior to the scan conversion.

The data processor 130 may detect an actual distance between pixels of the ultrasound image by using the scan line information and acquire information regarding a direction or strength of the edge based on the actual distance between pixels. According to an exemplary embodiment, the strength of the edge may indicate a brightness value difference between regions divided based on the detected edge.

The data processor 130 may reduce speckles by processing the regions divided based on the detected edge according to different references. That is, the data processor 130 may process the ultrasound image along the detected direction of the edge so that an edge of a region of which the edge having a high strength is detected is more vividly identified. In addition, the data processor 130 may process the ultrasound data along all directions regardless of the direction of the edge for a region of which an edge having a low strength is detected. Accordingly, the data processor 130 may process the ultrasound data so that a region including the edge is more vivid and a region without the edge is smooth.

According to an exemplary embodiment, the data processor 130 may detect an edge by setting an asymmetrical window of a predetermined size. According to another exemplary embodiment of the present invention, the data processor 130 may detect an edge by down-sampling the ultrasound data. These embodiments will be described in detail below with reference to FIGS. 7 and 8.

The scan converter 140 performs scan conversion of ultrasound data from which speckle noise has been reduced by the data processor 130. The scan converter 140 may generate a rectangular or fanwise ultrasound image by scan-converting the ultrasound data represented in an orthogonal coordinate system or in a polar coordinate system.

According to an exemplary embodiment, the scan converter 140 may scan-convert the ultrasound image by using the scan line information. That is, the scan converter 140 may convert the ultrasound image by performing an interpolation process according to a shape of a scan line indicated by the scan line information.

The display 150 may provide visual information to the user by displaying ultrasound diagnosis information, an ultrasound image, or a graphic user interface (GUI) related to a setup function of the apparatus 100. For example, the display 150 may display on a screen various types of images, such as a 2D or 3D ultrasound image, a Doppler image, and so forth.

The display 150 may be at least one of a liquid crystal display, a thin film transistor-liquid crystal display, an organic light-emitting diode, a flexible display, and a 3D display. In addition, the apparatus 100 may include two or more displays 150, according to a form of implementation.

According to an exemplary embodiment, the display 150 may include a user input (not shown) and a touch screen of a layer structure. That is, the display 150 may be used as both an output device and an input device, and in this case, the display 150 may receive a touch input using a stylus pen or touch by a part, such as a finger, of the human body.

A method of processing ultrasound data will now be described with reference to FIG. 3 by using the components included in the apparatus 100. FIG. 3 is a flowchart which illustrates a method of processing ultrasound data, according to an exemplary embodiment. The method illustrated in FIG. 3 includes operations sequentially processed by the data acquisition device 110, the scan line information acquisition device 120, the data processor 130, the scan converter 140, and the display 150 included in the apparatus 100 of FIG. 2. Thus, although omitted hereinafter, the descriptions related to the components of FIG. 2 may be applied to the method of FIG. 3.

In operation 210, the apparatus 100 receives an ultrasound signal. In response to a transmission ultrasound signal being reflected by an object, the apparatus 100 receives a reflected ultrasound signal.

In operation 220, the apparatus 100 generates ultrasound data. The apparatus 100 may generate the ultrasound data by analyzing the received ultrasound signal.

As described in FIG. 2, in operations 210 and 220, the apparatus 100 may receive ultrasound data from an external device instead of directly transmitting and receiving the ultrasound signal.

In operation 230, the apparatus 100 acquires scan line information. The apparatus 100 may acquire the scan line information from a probe which has transmitted and received the ultrasound signal or may extract the scan line information from the ultrasound data.

In operation 240, the apparatus 100 reduces speckles of the ultrasound image. The apparatus 100 may reduce speckles before scan conversion by using the scan line information acquired in operation 230. As described in FIG. 2, a detailed operation of reducing speckle noise in the apparatus 100 will be described with reference to FIG. 4.

In operation 250, the apparatus 100 scan-converts the ultrasound image from which speckles have been reduced. The apparatus 100 may generate an ultrasound image by scan-converting the ultrasound image with reduced speckles.

In operation 260, the apparatus 100 displays an ultrasound image of which image quality has been improved prior to the scan conversion.

According to the apparatus 100 and the method described above, a speckle pattern may be efficiently detected by detecting an edge included in the ultrasound image by using the scan line information prior to the scan conversion. Accordingly, speckles may be reduced prior to the scan conversion, thereby improving image quality of the scan-converted ultrasound image.

FIG. 4 is a flowchart which illustrates a method of processing ultrasound data, according to another exemplary embodiment. FIG. 4 illustrates another exemplary embodiment of the speckle noise reduction in operation 240 of FIG. 3.

In operation 242, the apparatus 100 detects an edge of the ultrasound image by using the scan line information. The apparatus 100 may acquire information regarding a distance between scan lines, a location on a scan line where the ultrasound signal is focused, and so forth from the scan line information and may accurately detect an edge included in the ultrasound image by utilizing the acquired information. As described in FIG. 2, the detected edge may indicate an edge of a structure of a bone, blood vessel, tissue, organ, or the like included in the ultrasound data.

In operation 242, the apparatus 100 may detect at least one of a strength and a direction of the edge. According to an exemplary embodiment, the strength of the edge may indicate a brightness value difference between regions divided based on the edge. The direction of the edge may indicate an oriented direction of the edge dividing the regions.

In operation 244, the apparatus 100 divides regions of the ultrasound image based on the edge detected in operation 242. The apparatus 100 may use information regarding the strength and direction of the edge described in operation 242 when dividing the regions, and a detailed embodiment thereof will be described with reference to FIG. 6.

In operation 246, the apparatus 100 processes the ultrasound image for each divided region. The apparatus 100 may process the ultrasound image of the regions divided based on the edge in operation 244 according to different references.

For example, the apparatus 100 may process the ultrasound image to be vividly viewed in the edge direction for a region of which a strength of the edge is high. In addition, the apparatus 100 may process the ultrasound image to be smoothly viewed in all directions for a region of which the strength of the edge is low.

Thereafter, in operation 250, the apparatus 100 may generate an ultrasound image by scan-converting the ultrasound image processed in operation 246.

FIG. 5 illustrates a scan conversion process according to an exemplary embodiment. The left image of FIG. 5 shows ultrasound image 510 prior to scan conversion, and the right image of FIG. 5 shows an ultrasound image 520 obtained by scan-converting the ultrasound image 510.

The apparatus 100 generates the ultrasound image 510 by using an ultrasound signal focused along a plurality of scan lines 530. In the exemplary embodiment illustrated in FIG. 5, a horizontal axis of the ultrasound image 510 indicates scan lines, and a vertical axis thereof indicates a depth axis of an object. In the exemplary embodiment illustrated in FIG. 5, the ultrasound image 510 includes three structures 512, 514, and 516. The structures 512, 514, and 516 may include bones, blood vessels, organs, tissues, and so forth of the object.

The ultrasound image 520 obtained by scan-converting the ultrasound image 510 includes structures 522, 524, and 526 which respectively correspond to the structures 512, 514, and 516 of the ultrasound data image. The structures 522, 524, and 526 of the ultrasound image 520 appear as if they were spread out in left and right directions (a lateral direction) due to the scan conversion of the ultrasound image 510, based on scan line information.

In the ultrasound image 520 in a fan shape, a length of an upper part in the lateral direction is different from a length of a lower part in the lateral direction. Accordingly, even though speckles have the same size in the ultrasound image 510, the speckles may have different sizes in the ultrasound image 520 according to locations of the speckles in a depth axis direction. That is, a speckle located at an upper part of the ultrasound image 520 and a speckle located at a lower part thereof may have the same size and shape in the ultrasound image 510 but may be have different sizes and shapes in the ultrasound image 520. For example, the speckle located at the upper part may appear small and close to other speckles, and the speckle located at the lower part may be appear long in the lateral direction.

The apparatus 100 may improve image quality of the ultrasound image 520 through various algorithms for reducing speckle noise. However, due to the shape of the ultrasound image 520, the apparatus 100 may not efficiently remove noise when reducing speckles in the scan-converted ultrasound image 520.

Meanwhile, speckles in the ultrasound image 510 have a constant shape and pattern regardless of locations thereof. Thus, the apparatus 100 may conveniently and efficiently improve image quality of the ultrasound image 520 when scan conversion of the ultrasound image 510 is performed after reducing speckles.

FIG. 6 illustrates a process of processing regions divided by a detected edge, according to an exemplary embodiment. Ultrasound image 610 shown in FIG. 6 includes a structure 615 drawn by a solid line. The apparatus 100 may detect an edge of the structure 615 by using scan line information.

The apparatus 100 may detect a strength and a direction of the edge of the structure 615. That is, the apparatus 100 may detect an oriented direction of the edge drawn by a solid line or a brightness value difference between regions divided based on the edge.

The apparatus 100 identifies the regions based on the detected edge. According to exemplary embodiment, the apparatus 100 may identify an inside region 620, an edge region 625, and an outside region 630 of the structure 615 based on the edge drawn by a solid line.

The apparatus 100 may process the inside region 620, the edge region 625, and the outside region 630 according to different references. That is, the apparatus 100 may differently process the inside region 620, the edge region 625, and the outside region 630 when reducing speckles of the ultrasound image 610.

According to an exemplary embodiment, the apparatus 100 may process the ultrasound image 610 by taking the detected direction or strength of the edge of the structure 615 into account. Alternatively, the apparatus 100 may take both the direction and strength of the edge into account. For example, the apparatus 100 may process the ultrasound image 610 to be smooth in all directions in the inside region 620 and the outside region 630 that are apart by a predetermined distance from the edge of the structure 615. To the contrary, the apparatus 100 may process the ultrasound image 610 for the edge region 625 in which the edge of the structure 615 is located so that the edge is more vivid.

According to the exemplary embodiment described above, the apparatus 100 may increase a difference in a gradient direction for the edge region 625 from which an edge in the ultrasound image 610 is detected in order for the edge to be more vivid. To the contrary, the apparatus 100 may process the ultrasound image 610 for the inside region 620 and the outside region 630 from which no edge is detected, i.e., having weak directivity, so that the inside region 620 and the outside region 630 are smooth in all directions. Accordingly, the apparatus 100 may easily detect and remove speckles of the ultrasound image 610.

FIG. 7 illustrates a process of setting a window to be applied to ultrasound data 710, according to an exemplary embodiment. The left image of FIG. 7 indicates the ultrasound image 710 prior to the scan conversion, and the right image of FIG. 7 indicates an ultrasound image subsequent to the scan conversion.

The apparatus 100 may determine a window to be applied to the ultrasound image 710. The window is a group of adjacent pixels, and the apparatus 100 may detect an edge by applying the window to the ultrasound image 710. According to an exemplary embodiment, the window has predetermined lengths in horizontal and vertical directions, wherein the horizontal direction of the window may correspond to a scan line, and the vertical direction thereof may correspond to a depth of an object.

In the left image of FIG. 7, a window 735 of a 3×3 size in the lower right consists of a total of nine pixels including a pixel 730 and eight pixels spatially adjacent to the pixel 730. A window 745 of a 3×5 size in the left consists of a total of fifteen pixels including a pixel 740. In FIG. 7, the horizontal direction of each of the windows 735 and 745 matches a plurality of scan lines 720. The windows 735 and 745 are respectively shown as windows 765 and 775 in a scan-converted ultrasound image 750. Similarly, the pixels 730 and 740 in the ultrasound image 710 are shown as pixels 760 and 770 in the ultrasound image 750, respectively.

The apparatus 100 may detect an edge by applying the windows 735 and 745 to the ultrasound image 710. That is, although the apparatus 100 may detect the edge from the whole region at once, the apparatus 100 may detect the edge by setting a window of a predetermined size and subsequently moving the window.

The apparatus 100 may determine a size of the window by using scan line information. According to an exemplary embodiment, the apparatus 100 may set the window 735 of a 3×3 size regardless of the scan line information. According to another exemplary embodiment, the apparatus 100 may determine horizontal and vertical lengths of the window 745 to be asymmetrical by using the scan line information. That is, the apparatus 100 may determine a size of the window according to a ratio of horizontal resolution to vertical resolution. For example, the apparatus 100 may determine a size of the window to have an asymmetrical ratio such as 3×5, 3×7, or the like.

FIG. 8 illustrates a process of down-sampling and processing ultrasound image 810, according to an exemplary embodiment. The left image of FIG. 8 indicates the ultrasound image 810 prior to scan conversion, and the right image of FIG. 8 indicates down-sampled ultrasound image 830.

For the ultrasound image 810 having high resolution in the vertical direction, the apparatus 100 may down-sample a vertical direction of the ultrasound image 810 before detecting an edge from the ultrasound image 810. As an example of the down-sampling, the apparatus 100 may perform a decimation process of the vertical direction of the ultrasound image 810.

A down-sampling ratio of the ultrasound image 810 in the apparatus 100 may be determined by the apparatus 100 according to a difference between resolutions of the ultrasound image 810 in horizontal and vertical directions. Alternatively, the apparatus 100 may receive a user input and may down-sample the ultrasound image 810, according to a ratio set by the user.

For the right image of FIG. 8, the apparatus 100 performs speckle reduction 840 for the down-sampled ultrasound image 830. Thereafter, the apparatus 100 may generate an ultrasound image by performing scan conversion 850 for ultrasound data of which image quality has been improved.

The apparatus 100 may quickly and efficiently perform the speckle reduction 840 by down-sampling the ultrasound image 810 before performing the speckle reduction 840 by detecting an edge of the ultrasound image 810.

The exemplary embodiments described with reference to FIGS. 7 and 8 are merely used to perform an image quality improving process for ultrasound image, and other various methods and algorithms may be used.

The methods of the exemplary embodiments can be written as computer programs and can be implemented in general-use digital computers that execute the programs using a non-transitory computer-readable recording medium. In addition, a data structure of data used in the methods can be recorded in a non-transitory computer-readable recording medium in various ways. It should be understood that program storage devices, which may be used to describe a storage device including executable computer codes for executing the methods of the exemplary embodiments, do not include temporary objects, such as carrier waves and signals. Examples of the non-transitory computer-readable recording medium include storage media such as magnetic storage media (e.g., ROM, floppy disks, hard disks, etc.) and optical recording media (e.g., CD-ROMs, or DVDs).

According to the apparatus and method for processing ultrasound image data, image quality of an ultrasound image may be efficiently improved by performing an image quality improving process prior to scan conversion of the ultrasound image. In addition, since speckles can be removed before information related to a high frequency band is damaged in a scan conversion process of the ultrasound image, the performance of removing speckles may be improved by distinguishing an ultrasound signal from noise.

In addition, regardless of an asymmetrical shape of the ultrasound image and the locations of speckles, speckles in similar shapes and patterns may be easily detected and removed. Furthermore, even though the ultrasound image is asymmetrical, a speckle removing process for an edge portion of the ultrasound image may be efficiently performed.

It will be understood by one of ordinary skill in the art that various changes in form and details may be made therein without departing from the spirit and scope of the exemplary embodiments as defined by the following claims. The exemplary embodiments should be considered in a descriptive sense only and not for purposes of limitation. Therefore, the scope of the exemplary embodiments is defined not by the detailed description of the but by the appended claims, and all differences within the scope will be construed as being included in the present invention.

What is claimed is:

1. A method of processing ultrasound data, the method comprising:
acquiring an ultrasound image related to an object by applying an ultrasound signal to the object;
acquiring scan line information related to the ultrasound signal applied to the object; and
reducing speckles of the ultrasound image by using the scan line information prior to scan conversion of the ultrasound image.

2. The method of claim 1, wherein the reducing comprises detecting an edge included in the ultrasound image by using the scan line information.

3. The method of claim 2, wherein the acquiring scan line information comprises acquiring at least one of a strength and a direction of the detected edge.

4. The method of claim 2, wherein the reducing comprises processing ultrasound image in regions divided by the detected edge based on different references.

5. The method of claim 4, wherein the processing comprises processing the ultrasound image in the divided regions based on the direction of the detected edge.

6. The method of claim 2, wherein the detecting comprises:
determining an asymmetrical window of a predetermined size to be applied to the ultrasound image based on the scan line information; and
detecting the edge by using the window.

7. The method of claim 6, wherein the determining of the asymmetrical window comprises determining a horizontal length and a vertical length of the asymmetrical window.

8. The method of claim 2, wherein the detecting comprises:
down-sampling the ultrasound image based on the scan line information; and
detecting an edge of the down-sampled ultrasound image.

9. The method of claim 1, further comprising:
scan-converting the ultrasound image from which the speckles have been reduced; and
displaying a scan-converted ultrasound image.

10. An apparatus for processing ultrasound data, the apparatus comprising:
a data acquisition device configured to acquire an ultrasound image related to an object by applying an ultrasound signal to the object;
a scan line information acquisition device configured to acquire scan line information related to the ultrasound signal applied to the object; and
a data processor configured to reduce speckles of the ultrasound image by using the scan line information prior to scan conversion of the ultrasound image.

11. The apparatus of claim 10, wherein the data processor is configured to detect an edge included in the ultrasound image by using the scan line information.

12. The apparatus of claim 11, wherein the data processor is configured to acquire at least one of a strength and a direction of the detected edge.

13. The apparatus of claim 11, wherein the data processor is configured to process an ultrasound image in regions divided by the detected edge based on different references.

14. The apparatus of claim 13, wherein the data processor is configured to process the ultrasound image in the divided regions based on the direction of the detected edge.

15. The apparatus of claim 11, wherein the data processor is configured to determine an asymmetrical window of a predetermined size to be applied to the ultrasound image based on the scan line information and detects the edge by using the asymmetrical window.

16. The apparatus of claim 15, wherein the data processor is configured to determine a horizontal length and a vertical length of the asymmetrical window.

17. The apparatus of claim 11, wherein the data processor is configured to down-sample the ultrasound image based on the scan line information and detects an edge of the down-sampled ultrasound image.

18. The apparatus of claim 11, further comprising:
a scan converter configured to scan-convert a ultrasound image from which the speckles have been reduced; and
a display configured to display a scan-converted ultrasound image.

19. A non-transitory computer-readable storage medium having stored therein program instructions, which when executed by a processor of a computer, cause the computer to perform the method of claim 1.
